(19) United States
(12) Patent Application Publication
(10) Pub. No.: US 2014/0370504 A1
(43) Pub. Date: Dec. 18, 2014

(54) METHOD FOR DETECTING GENETIC VARIATION

(71) Applicants: Shengpei Chen, Shenzhen (CN); Chunlei Zhang, Shenzhen (CN); Fang Chen, Shenzhen (CN); Weiwei Xie, Shenzhen (CN); Xiaoyu Pan, Shenzhen (CN); Jian Wang, Shenzhen (CN); Jun Wang, Shenzhen (CN); Huanming Yang, Shenzhen (CN); Xiuqing Zhang, Shenzhen (CN)

(72) Inventors: Shengpei Chen, Shenzhen (CN); Chunlei Zhang, Shenzhen (CN); Fang Chen, Shenzhen (CN); Weiwei Xie, Shenzhen (CN); Xiaoyu Pan, Shenzhen (CN); Jian Wang, Shenzhen (CN); Jun Wang, Shenzhen (CN); Huanming Yang, Shenzhen (CN); Xiuqing Zhang, Shenzhen (CN)

(73) Assignee: BGI DIAGNOSIS CO., LTD., Shenzhen (CN)

(21) Appl. No.: 14/369,615

(22) PCT Filed: Dec. 31, 2011
(86) PCT No.: PCT/CN2011/002244

§ 371 Date: Jun. 27, 2014

(51) Int. Cl. G06F 19/22; G06F 17/18; C12Q 1/68

(52) U.S. Cl. CPC G06F 19/22; C12Q 1/6869; G06F 17/18
USPC 435/6.11; 702/20

(57) ABSTRACT

The present invention relates to a method for detecting genetic variation, comprising the following steps: acquiring reads from a test sample; aligning said reads with a reference genome sequence; dividing said reference genome sequence into windows, calculating the number of said reads which are aligned to each window, and acquiring the statistic for each window on the basis of the number of said reads; and for a fragment of the reference genome sequence, acquiring the genetic variation sites on the basis of the change in the statistics of all the windows thereon in the fragment of the reference genome sequence.

### TECHNICAL FIELD

The present invention relates to the field of genetic variation detection, and in particular, to the detection of a copy number variation, e.g., microdeletion/microduplication and aneuploidy.

### BACKGROUND ART

A copy number variation (CNV) refers to a submicroscopic mutation of a DNA fragment in a range from kb to Mb, which is marked by the increase or decrease in the copy number. Research on the relationship between copy number variation and disease has a long history. For some germline mutation copy number variations (i.e. copy number variations generated due to variations of a fetus itself, which are absent in both of the parents), it is believed that the larger a fragment is, the easier a congenital anomaly occurs, and for example, chromosomal aneuploidy diseases (e.g. T21, T18 etc.) and chromosomal microdeletion/microduplication syndromes are all recognized germline mutation copy number variation related diseases.

Human chromosomal microdeletion/microduplication syndromes are a disease type of complex and changeable phenotypes caused by the occurrence of micro-fragment deletions or duplications, i.e., copy number variations in DNA fragments, on human chromosomes with a relatively high incidence in perinatal infants and neonatal infants, and can lead to serious diseases and abnormalities, e.g., congenital heart disease or heart malformation, serious growth retardation, appearance or limb deformity, etc. In addition, the microdeletion syndromes are also one of the main reasons causing mental retardation besides Down's syndrome and fragile X syndrome [symbol] Knight S J L (ed): Genetics of Mental Retardation. Monogr Hum Genet. Basel, Karger, 2010, vol 18, pp 101-113 (DOI: 10.1159/000287600) [symbol] . In recent years, the congenital heart disease topping the list in the statistics for the incidence of birth defects and the mental retardation, cerebral palsy and congenital deafness which are top-ranked in outpatient clinics for genetic counseling and diagnosis are all related to the microdeletion syndromes. Common microdeletion syndromes include 22q11 microdeletion syndrome, cri du chat syndrome, Angelman syndrome, AZF deletion, etc.

Although the incidence of each microdeletion syndrome is very low, wherein the incidences of the relatively common 22q11 microdeletion syndrome, cri du chat syndrome, Angelman syndrome, Miller-Dieker syndrome, etc. are 1:4,000 (live births), 1:50,000, 1:10,000 and 1:12,000 respectively. Due to the limitation by clinical detection techniques, a large number of patients with microdeletion syndromes cannot be detected in prenatal screening and prenatal diagnosis. And even when a reason is looked for retrospectively after the occurrence of typical clinical characterizations months or even years after the birth of an infant, the cause of the disease also cannot be diagnosed due to the limitation by the detection techniques (https://decipher.sanger.ac.uk/syndromes). Because a radical cure cannot be effected for some types of microdeletion syndromes with the death within months or years after the birth, a heavy mental and economic burden is brought to the society and families. According to incomplete statistics, patients with “happy puppet syndrome” (Angelman syndrome) worldwide have reached 15 thousand, and the numbers of patients with the other types of chromosomal microdeletion syndromes are also increasing year by year. Thus, the pre-pregnancy detection of chromosomal microdeletions/microduplications performed on clinically suspected patients and parents with a related abnormal pregnancy history is conducive to providing genetic counseling and providing a basis for clinical decision making. The early prenatal diagnosis during pregnancy can effectively prevent the birth of an infant patient or provide a basis for providing a treatment approach for an infant patient after birth [symbol] Bretelle F, et al. Prenatal and postnatal diagnosis of 22q11.2 deletion syndrome. Eur J Med Genet. 2010 November-December; 53(6):367-70 [symbol] .

However, this type of diseases cannot be detected by routine clinical methods such as the chromosome karyotyping method and the like (with a resolution of above 10 M) because of the micro level of variation in chromosome [symbol] Malcolm S. Microdeletion and microduplication syndromes. Prenat Diagn. 1996 December; 16(13):1213-9 [symbol] . Currently, for the prenatal diagnosis of the microdeletion/microduplication syndromes, methods of invasive fetal amniotic fluid or other tissues are mainly adopted to perform the molecular diagnosis. Currently, invasive molecular diagnostic methods mainly include high-resolution chromosome karyotyping, FISH (fluorescence in situ hybridization), Array CGH (comparative genomic hybridization), MLPA (multiplex ligation-dependent probe amplification technique), PCR and the like. Among them, the FISH examination is used as a gold standard for genetic diagnosis which can effectively detect most of deletions of chromosomal fragments. However, because invasive sampling needs a certain surgery or cell culture, from the point of time efficiency and resource consumption, same is suitable for acting as a diagnostic indicator, but not a method for universal clinical screening.

In terms of non-invasive screening methods for the microdeletion/microduplication syndromes, there are also some attempts. For example, in a study on the non-invasive detection of the microdeletion syndromes in a fetus published in November 2011, the researchers performed high depth sequencing on plasma of the mother during pregnancy, generated about 243 million short reads, and detected a microdeletion of around 4 Mb from 12p11.22 to 12p12.1 in the fetus [symbol] David Peters, et al. Noninvasive Prenatal Diagnosis of a Fetal Microdeletion Syndrome. N Engl J Med 2011; 365:1847-1848 [symbol] . However, the generation of such a large amount of data is not suitable for clinical use in terms of either resource consumption or time efficiency.

It can be known from the combination of the above-mentioned contents that currently, among the prenatal examination methods for chromosomal microdeletion/microduplication syndromes, there is still no feasible universal screening method. A new credible screening method for copy number variation in a fetus is needed in this field, so as to identity known sites and to explore unknown sites discoverably.

### SUMMARY OF THE INVENTION

With the continuous development of the high-throughput sequencing technique and the continuous reduction in the sequencing cost, studies on sequencing techniques in prenatal screening make analysis of screening of chromosomal copy number variation and aneuploidy and other genetic variations, and in particular, fetal aneuploidy chromosomal variation, by high-throughput sequencing, be more and more widely applied. In order to detect genetic variation, the present invention designs a genetic variation screening method based on the high-throughput sequencing technique which can use the detection of copy number variation and aneuploidy and other genetic variations and has the features of high throughput, high specificity and accurate location. The method of the present invention includes the steps of acquiring a test sample and extracting DNA, performing high-throughput sequencing and analyzing the obtained data to obtain a detection result.

The present invention provides a method for detecting genetic variation, comprising the following steps:

1) acquiring reads from a test sample, wherein the fragment of said reads, for example, can be 25-100 nt in length, and the fragment number of said reads can be at least 1 million;

2) aligning said reads with a reference genome sequence;

3) dividing said reference genome sequence into windows, calculating the number of reads which are aligned to each window, and acquiring the statistic for each window on the basis of the number of said reads;

4) and for a fragment of the reference genome sequence, on the basis of the change in the statistics of all the windows thereon in the fragment of the reference genome sequence, acquiring positions where a significant change occurs in statistics of the windows on both sides, these positions being positions where genetic variation sites of the test sample are on the reference genome sequence.

In an embodiment, said genetic variation site in the method of the present invention is the median point between an inflection point where said statistic turns from ascending to descending and the next same inflection point, and there is at least 50, at least 70, at least 100, preferably 100 window lengths between two genetic variation sites; and the above-mentioned site, inflection point and median point refer to a chromosomal position corresponding to a window corresponding to the statistic, and can be represented by the starting point, the midpoint, the end point and any other position of the window.

In a particular embodiment, the method of the present invention further comprises the following step:

5) screening the genetic variation sites to obtain post-screening genetic variation sites.

For example, the above-mentioned step 5) is:

for two fragments of the sequence between each genetic variation site to the preceding genetic variation site and to the following genetic variation site, performing statistics on the difference between two numerical groups consisting of statistics of windows contained in said two fragments of the sequence, and removing the genetic variation site whose significance value of difference is maximum and greater than a preset threshold; and repeating the above-mentioned process, until the significance values of difference of the genetic variation sites are all smaller than the preset threshold,

wherein said significance of difference, e.g., can be performed by the run test, removing the genetic variation site whose significance value in the run test is maximum and greater than the preset threshold; and repeating the above-mentioned process, until the significance values of the genetic variation sites in the run test are all smaller than the preset threshold.

In an embodiment, the preset threshold used in the above-mentioned step 5) can be obtained by the following steps:

a) acquiring the genetic variation sites according to the method of the present invention by substituting the test sample with a control sample;

b) for two fragments of the sequence between each genetic variation site to the preceding genetic variation site and to the following genetic variation site, performing statistics on the difference between two numerical groups consisting of statistics of windows contained in said two fragments of the sequence, and removing the genetic variation site which is the least significant;

c) and repeating the above-mentioned step b), until the number of remaining candidate breakpoints is equal to the expected value N_{c}, wherein N_{c}=L_{c}/T, L_{c} is the length of the genome sequence, the theoretical ultimate precision T is the fragment size which can be detected theoretically, the theoretical ultimate precision T=W+S*N when the average of the window sizes is W, the sliding length of the windows is S and the number of each window group in the run test is N, and among significance values of all the remaining candidate breakpoints, the minimum is the significance threshold.

The present invention also provides a method for detecting genetic variation, comprising the following steps:

1) acquiring the genetic variation sites on a fragment of the reference genome sequence according to the method of the present invention;

2) and a step of performing a confidence-based selection on fragments between said genetic variation sites.

In an embodiment of the present invention,

the step of confidence-based selection in the above-mentioned step 2) is:

i) calculating the distribution probability of the statistics through the distribution pattern of the statistics for the windows, and setting a threshold;

ii) and comparing the average of the statistics of windows in the fragment between post-screening genetic variation sites with said threshold, and determining whether the fragment between the genetic sites is anomalous on the basis of the comparison result.

In another embodiment of the present invention, the step of confidence-based selection in the above-mentioned step 2) is:

i) calculating the distribution probability of the statistics through the distribution pattern of the statistics for the windows, and setting a first threshold and a second threshold;

ii) and comparing the average of the statistics of windows in the fragment between post-screening genetic variation sites with said first threshold and second threshold,

wherein, if the statistics for windows in the fragment are smaller than the first threshold, the fragment is a fragment deletion, and if same are greater than the second threshold, the fragment is a fragment duplication,

wherein said first threshold is a value of the statistic where the cumulative probability of the occurrence of the statistic is less than or equal to 0.1, preferably less than or equal to 0.01, most preferably 0.05, and/or said second threshold can be a value of the statistic where the cumulative probability of the occurrence of the statistic is greater than or equal to 0.9, preferably greater than or equal to 0.99, most preferably 0.95.

The present invention also provides a computer-readable medium, carrying a series of executable codes, which can execute the method of genetic detection of the present invention.

The present invention also provides a method for detecting fetal genetic variation, comprising the following steps:

acquiring a maternal sample containing fetal nucleic acid;

sequencing said maternal sample;

and a step of detecting the genetic variation using the method of any of claims 1-16.

In an embodiment of the present invention, said maternal sample is maternal peripheral blood.

The superiority of the present invention, compared with the current methods for detecting genetic variation, mainly includes the following points:

(1) Being clinically feasible: we use only around 5 M of sequencing data, and can detect around 5 Mb of CNV fragments, while a reported method used that of nearly 243 M, therefore, our method reduces the cost and time consumption of data generation greatly.

(2) Being extensible: we can increase the precision by expanding the number of control groups besides by increasing the amount of sequencing, so as to reduce the pressure on the starting amount of DNA.

(3) Being more stable and more comprehensive: there is no detail of the operation itself pointed out clearly in reported articles, while the present invention designs the correction of data groups, the preference for fragmentation conditions and various other aspects.

### DESCRIPTION OF THE DRAWINGS

FIG. 1 is a brief flowchart on the genetic variation analysis of chromosomes in an example of the present invention.

FIG. 2A is a digital chromosomal karyogram of S67.

FIG. 2B is a digital chromosomal karyogram of S10.

FIG. 2C is a digital chromosomal karyogram of S14.

FIG. 2D is a digital chromosomal karyogram of S18.

FIG. 2E is a digital chromosomal karyogram of S49.

FIG. 2F is a digital chromosomal karyogram of S55.

FIG. 2G is a digital chromosomal karyogram of S82.

FIG. 2H is a digital chromosomal karyogram of S103.

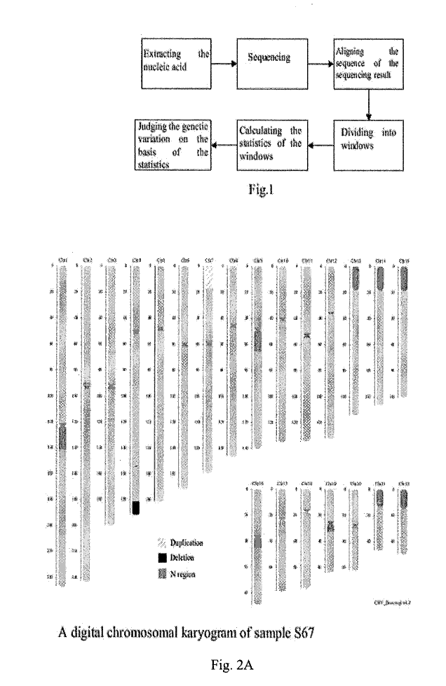

### DESCRIPTION OF THE TABLES

Table 1 is a list of CNV results of all samples in the implementation case.

Table 2 shows aCGH and karyotyping detection results of all samples in the implementation case.

Table 3 shows the test results in the present implementation case and the results of standard karyotyping detection.

### Embodiments

According to the examples of the present invention, the test sample is a sample containing nucleic acid, and the type of the nucleic acid is not particularly limited, which can be deoxyribonucleic acid (DNA), and can also be ribonucleic acid (RNA), preferably DNA. It would be understood by those skilled in the art that for RNA, same can be converted by conventional means into DNA with a corresponding sequence to perform subsequent detection and analysis. In addition, the property of the test sample is not particularly limited either. According to some examples of the present invention, a genomic DNA sample can be adopted, and a part of genomic DNA can also be adopted as the test sample. According to the examples of the present invention, the source of the test sample is not particularly limited. According to the examples of the present invention, a sample from a pregnant woman can be adopted as the test sample, from which a nucleic acid sample containing fetal genetic information can thereby be extracted, and then the fetal genetic information and physiological state can be detected and analyzed. According to the examples of the present invention, examples of a sample from a pregnant woman which can be used include, but are not limited to, peripheral blood of the pregnant woman, urine of the pregnant woman, cervical fetal exfoliated trophoblastic cells of the pregnant woman, cervical mucus of the pregnant woman, and fetal nucleated red blood cells. The inventors found that through the extraction of the nucleic acid sample from the above-mentioned sample of a pregnant woman, the genetic variation in the genome of a fetus can be analyzed effectively to realize the non-invasive prenatal diagnosis or detection on the fetus. It is an advantage of the present invention that non-invasive detection of fetal genetic variation can be performed, for example, said sample is peripheral blood of a pregnant woman, moreover, the method of the present invention is also suitable for invasive detection, for example, said sample can be from fetal cord blood; said tissue can be placental tissue or chorionic tissue; and said cells can be uncultured or cultured amniotic fluid cells and villus progenitor cells. In the present invention, a subject to be tested and a normal subject are of the same species. Furthermore, the variation detection of the present invention is not necessarily used for diagnosis of diseases or related purposes, because with the presence of polymorphism, the presence of some variations relative to a reference genome does not represent the risk of suffering from a disease or the state of health. Thus, the variation detection of the present invention can be simply for use in scientific research on genetic polymorphism.

In the present invention, a control sample is against the test sample. For example, in a method related to the detection of a disease, the control sample refers to a normal sample. For example, in an embodiment of the present invention, the test sample is maternal peripheral blood, and the corresponding control sample is peripheral blood of a normal mother conceiving a normal fetus.

According to the examples of the present invention, the method and apparatus for extracting the nucleic acid sample from the test sample is not particularly limited either, and commercialized nucleic acid extraction kits can be adopted to perform same.

In the method of the present invention, said windows have the same number of reference unique reads. The reference unique reads refer to a chromosomal fragment with a unique sequence, this fragment can be definitely located at a single chromosomal position, and the chromosomal reference unique reads can be constructed based on a disclosed chromosomal reference genome sequence, e.g., hg18 or hg19. A process for acquiring the reference unique reads generally include the steps of cutting the reference genome into reads of any fixed length, aligning these reads back to the reference genome, and selecting the reads which are aligned to the reference genome uniquely as the reference unique reads. Said fixed length depends on the lengths of sequences in the sequencing result by a sequencer, referring to the average length for detail. The lengths in the sequencing results obtained by different sequencers are different, and specifically for each run of sequencing, the lengths in the sequencing results may also be different, and there are certain subjective and experience factors existing in the selection of the length.

In an example of the present invention, the length of the reference unique reads is selected according to the actual lengths of sequences in the sequencing result, e.g. 25-100 bp, and for the illumina/Solexa system, e.g. optionally 50 bp, and then the number of reference unique reads contained in each window is controlled at 800,000-900,000. In the method of the present invention, said windows can have an overlap or have no overlap therebetween. In an example of the present invention, the distance between adjacent windows is 1-100 kb, preferably 5-20 kb, more preferably 10 kb. This distance can be adjusted according to the DNA abundance in the fetal sample. The principle of the adjustment is that each window corresponds to one statistic and one chromosomal position, which also means that the distance between windows determines the precision of detection. The higher the precision is, the higher the maternal derived background is, and the more difficult the discrimination of sources of genetic variations is.

In the method of the present invention, said statistic can be the number of reads itself, but preferably is a statistic after error correction (e.g. GC correction) and/or data standardization, the purpose of which is that the statistic meets a common distribution in the statistics, e.g. the normal or standard normal distribution. The subsequent statistical analysis of the statistics can thus be facilitated. In an example of the present invention, the standardization processing against the average number of reads of all the windows is performed. In an example of the present invention, the standardization includes a process for evaluating the Z value hereinafter. In an embodiment, said statistic approximately fits normal distribution obtained by the standardization processing on the number of reads which are aligned to a window. In an embodiment, said standardization is based on the average number of reads which are aligned to all the windows. In an embodiment, said statistic is an approximate standard normal distribution statistic.

In the present invention, the reads refer to sequence fragments outputted by a sequencer, preferably about 25-100 nt.

In the present invention, said DNA molecules can be acquired using the salting-out method, the column chromatography method, the magnetic bead method, the SDS method and other routine DNA extraction methods, preferably using the magnetic bead method. The so-called magnetic bead method refers to for bare DNA molecules obtained after the blood, tissues or cells undergo the action of a cell lysis solution and proteinase K, using specific magnetic beads to perform reversible affinity adsorption on the DNA molecules, and after proteins, lipids and other impurities are removed by washing with a rinsing liquid, eluting the DNA molecules from the magnetic beads with a purification liquid. The magnetic beads are well known in the art, and are commercially available, e.g. from Tiangen.

In the present invention, generally, the direct sequencing of the DNA molecules obtained from the samples and subsequent steps can realize the purpose of the present invention, and the extracted DNA can be used for the subsequent steps without being processed. In some preferred embodiments, fragments with electrophoretic main bands concentrated in the size of 50-700 bp, preferably 100-500 bp, more preferably 150-300 bp, particularly about 200 bp, may only be studied. In some more preferred embodiments of the present invention, the DNA molecules can be broken into fragments with electrophoretic main bands concentrated in a certain size, e.g., 50-700 bp, preferably 100-500 bp, more preferably 150-300 bp, particularly near 200 bp, and then the subsequent steps are performed. The treatment of randomly breaking said DNA molecules can use enzyme digestion, atomization, ultrasound or the HydroShear method. Preferably, the ultrasound method is used, for example, the S-series of the Covaris Corporation (based on the AFA technique, wherein when the sound energy/mechanical energy released by a sensor passes through a DNA sample, gas is dissolved to form bubbles; after removing the energy, the bubbles burst and the ability to fracture DNA molecules is generated; through setting of a certain energy intensity and time interval and other conditions, the DNA molecules can be broken into sizes within a certain range; for example, for a specific principle and method, see the instructions for the S-series from the Covaris Corporation).

In the present invention, said breakpoint or candidate breakpoint is a potential or existing genetic variation site, and by convention, the site is expressed as the position on the reference genome. In the present invention, the two concepts, genetic variation site and breakpoint, are interchangeable in a particular case, and just different in expression, and may both be used to represent the position coordinate of a potential or definitely existing genetic variation on the reference genome in various stages.

In the present invention, the method of sequencing can be adopted to acquire the reads from the test sample, and said sequencing can be performed through any sequencing method, which includes, but is not limited to, the dideoxy chain-termination method; preferably a high-throughput sequencing method, which includes, but is not limited to, second-generation sequencing techniques or single molecule sequencing techniques (Rusk, Nicole (2009 Apr. 1). Cheap Third-Generation Sequencing. Nature Methods 6 (4): 2446 (4).

Platforms for said second-generation sequencing (Metzker M L. Sequencing technologies—the next generation. Nat Rev Genet. 2010 January; 11(1):31-46) include, but are not limited to, Illumina-Solexa (GATM, HiSeq2000TM, etc.), ABI-Solid and Roche-454 (pyrosequencing) sequencing platform; and platforms (techniques) for the single molecule sequencing include, but are not limited to, True Single Molecule DNA sequencing from the Helicos Corporation, single molecule real-time sequencing (SMRTTM) from the Pacific Biosciences Corporation, and nanopore sequencing technique from the Oxford Nanopore Technologies Corporation, etc.

The type of sequencing can be single-end sequencing and pair-end sequencing, and the sequencing length can be 50 bp, 90 bp or 100 bp. In an embodiment of the present invention, said sequencing platform is Illumina/Solexa, the type of sequencing is pair-end sequencing, and 100 bp sized DNA molecule sequence with the pair-end positional relationship is obtained.

In an embodiment of the present invention, the sequencing depth of sequencing can be determined according to the size of fetal chromosomal variation fragment to be detected, and the higher the sequencing depth is, the higher the detection sensitivity is, i.e., the smaller the detectable deletion and duplication fragment is. The sequencing depth can be 1-30×, i.e., the total amount of data is 1-30 times the length of the human genome, for example, in an embodiment of the present invention, the sequencing depth is 0.1×, i.e., 2 times (2.5×108 bp).

When the DNA molecules to be tested are from a plurality of test samples, a different tag sequence can be added to each sample, so as to discriminate the samples in the sequencing process (Micah Hamady, Jeffrey J Walker, J Kirk Harris et al. Error-correcting barcoded primers forpyrosequencing hundreds of samples in multiplex. Nature Methods, 2008, March, Vol. 5 No. 3), thereby realize simultaneous sequencing of the plurality of samples. The tag sequences are for discriminating different sequences, but will not affect other functions of the DNA molecules to which the tag sequences added. The length of a tag sequence can be 4-12 bp.

In an example of the present invention, said human genomic reference sequence is a human genomic reference sequence in the NCBI database. In an embodiment of the present invention, said human genome sequence is the human genomic reference sequence build 36 in the NCBI database (hg18; NCBI Build 36).

In the present invention, said alignment can be alignment with no mismatch allowed, and can also be alignment with 1 base mismatch allowed. The sequence alignment can be performed through any sequence alignment program, for example, the Short Oligonucleotide Analysis Package (SOAP) and the BWA (Burrows-Wheeler Aligner) alignment that are available to those skilled in the art, and the reads are aligned with the reference genome sequence to obtain the reads' positions on the reference genome. The sequence alignment can be performed using the default parameters provided by the program, or the parameters are selected by those skilled in the art according to the need. In an embodiment of the present invention, the alignment software used is SOAPaligner/soap2.

In the present invention, the algorithm of said software is a series of programs for detection of copy number variation in a fetus developed by the BGI institute in Shenzhen, which are collectively referred to as FCAPS. It can perform data correction, standardization and fragmentation on a test sample and a control set through data generated by the new-generation sequencing technique, and estimate the extent and size of copy number variations in a fetus.

In some particular embodiments of the method of the present invention, for step 1), acquiring reads from a test sample: after the extraction of plasma DNA from the test sample and the control sample according to the operation manual for Tiangen DP327-02 Kit, a library is constructed according to the modified Illumina/Solexa standard library construction flow. For details of the construction of a whole-genome sequencing library, see the directive rules provided by the manufacturer of the sequencer, e.g. the Illumina Corporation, for example, see Multiplexing Sample Preparation Guide (Part#1005361; February 2010) or Paired-End SamplePrep Guide (Part#1005063; February 2010) by the Illumina Corporation, which is incorporated herein by reference. In this process, adapters used for sequencing are added to both ends of the DNA molecules which are concentrated at 200 bp themselves, a different tag sequence is added to each sample, thereby data for a plurality of samples can be discriminated in data obtained by a single run of sequencing, and with the use of the second-generation sequencing method, Illumina/Solexa sequencing (other sequencing methods such as ABI/SOLiD can be used to achieve the same or similar effect), reads with a certain fragment size are obtained for each sample.

In some particular embodiments of the method of the present invention, for step 2) alignment: the reads in step 1) in the method of the present invention are SOAP2-aligned with the standard human genomic reference sequence in the NCBI database to obtain the positional information about the sequenced DNA sequence on the genome. For avoiding the disturbance to the CNV analysis by repeat sequences, only reads that are aligned with the human genomic reference sequence uniquely are selected for subsequent analysis.

In some particular embodiments of the method of the present invention, step 3), dividing into windows and acquiring statistics for the windows, comprises the following steps:

a) for the test sample and the control sample, providing windows with the length of w on the genomic reference sequence, calculating the GC content in each window and calculating the relative fragment number of reads falling into each window; and b) correcting and standardizing the relative fragment number of reads of the above-mentioned test sample against the relative fragment number of reads of the control sample.

In some particular embodiments of the method of the present invention, GC correction based on the control sample set is performed on the test sample: because a certain GC bias exists between/within sequencing batches, which makes a copy number deviation occur in the high GC region or in the low GC region in the genome, the corrected relative number of reads in each window obtained by the GC correction of sequencing data based on the control sample set can remove this bias and improve the precision of detection of copy number variation. The corrected relative number of reads in each window is standardized: copy number variation in a fetus is detected using plasma from the pregnant mother, and with the effect of the mother's DNA background, the variation in the fetus are relatively difficult to stand out, so it is demanded to reduce the noise of the mother's DNA background and amplify the signal of the copy number variation in the fetus through standardization. In an embodiment of the present invention, said GC correction comprises the following steps: a) acquiring reads which are aligned to each window according to the method of the present invention by substituting the test sample with a control sample, and calculating the relative number of the reads for each window; b) acquiring the functional relationship between the GC content of the reads which are aligned to each window and the relative number of the reads for said window; and c) for each window, using the GC content of reads of the test sample aligned to the window and the above-mentioned functional relationship, and by correcting the relative number of reads of the test sample for the window to obtain the corrected relative number of reads for the window.

In some particular embodiments of the method of the present invention, step 3), dividing into windows and acquiring statistics for the windows, comprises the following steps:

a) calculating the relative number of reads of the test sample and that of the control sample:

for the test sample and the control sample, providing windows with the length of w on the human genomic reference sequence, calculating the number of reads, r_{i,j}, falling in each window in step 2) in the method of the present invention, where the subscripts i and j represent the serial number of the window and the serial number of the sample respectively, and calculating the GC content, GC_{i,j}, of each window and calculating the relative number of reads,

$${R}_{i , j} = {log}_{2} ( \frac{{r}_{i , j}}{{\overline{r}}_{j}} ) ,$$

where the average number of reads is

$${\overline{r}}_{j} = \frac{1}{n} {\sum }_{i = 1}^{n} {r}_{i , j} ,$$

b) data correction and standardization:

\textcircled{1} in a coordinate system with the GC content as the abscissa and the relative number of reads R as the ordinate, performing linear fitting on R_{i,j} and GC_{i,j} of the control sample to obtain the slope aᵢ and the intercept bᵢ,

\textcircled{2} for each window of the test sample, calculating the corrected relative number of reads \tilde{R}_{i,j}=aᵢ×GC_{i,j}+bᵢ,

\textcircled{3} For each window of the test sample, calculating the statistic Z_{i,j}:

Z=(R_{i,j}−\tilde{R}_{i,j}−meanⱼ)/SDⱼ,

where

$${mean}_{j} = \frac{1}{n} {\sum }_{i = 1}^{n} ( {R}_{i , j} - {\tilde{R}}_{i , j} ) , \\ {SD}_{j} = \sqrt{\frac{1}{n - 1} {\sum }_{i = 1}^{n} {( {R}_{i , j} - {\tilde{R}}_{i , j} - {mean}_{j} )}^{2}} .$$

In some particular embodiments of the method of the present invention, acquiring positions where genetic variation sites of the test sample are on the reference genome sequence in step 4) is performed through the following steps:

\textcircled{1} initialization: for an end point of each window, if the change trend of the statistic Z is changed between the preceding window and the following window of the point, and the distance between the point and a previous point where the change trend of the statistic Z is changed between the preceding window and the following window is at least n windows (n is an integer of 10-500, preferably 50-300, e.g. 100), then the point is a candidate breakpoint, for example, the midpoint between that inflection point where the statistic Z turns from ascending to descending between the preceding window and the following window and the next same inflection point is a candidate breakpoint, or the midpoint between that inflection point where the statistic Z turns from descending to ascending between the preceding window and the following window and the next same inflection point is a candidate breakpoint bk (k=1, 2, . . . , s; s is an integer of >0);

\textcircled{2} optimal iteration: in order to study copy number variation or aneuploidy in a fragment of genome sequence, all sorted candidate breakpoints of the fragment of the genome sequence are recorded as B_{c}={b₁, b₂, . . . , bₛ}, wherein two fragments, one on the left and the other on the right, exist for each candidate breakpoint bₖ and said fragments are a region from the previous breakpoint to the breakpoint and a region from the breakpoint to the next breakpoint; the p value (pₖ) obtained by subjecting Z_{i,j} for all windows in these two fragments to a test (e.g., the run test which is a non-parametric test and using the uniform state of distribution after mixing of elements from two groups to evaluate the significance of difference between these two groups) is regarded as “the significance of bₖ as a breakpoint”; a candidate breakpoint with the maximum pₖ is excluded; and this step is repeated until all p values are smaller than the termination p value (p_{final}) for the genome sequence;

\textcircled{3} acquisition of the termination p value: in the test process, the above-mentioned steps a) to c) are performed by using another control sample as the test sample, for a fragment of a genome sequence, all sorted candidate breakpoints of the fragment of the genome sequence are recorded as B_{c}={b₁, b₂, . . . , bₛ}, wherein two windows, one on the left and the other on the right, exist for each candidate breakpoint bₖ; the p value (pₖ) obtained by subjecting all Z_{i,j} in these two windows to the run test is regarded as “the significance of bₖ as a breakpoint”; a candidate breakpoint with the maximum pₖ is excluded; the two windows on the left and right thereof are merged until the number of candidate breakpoints is equal to the expected value N_{c}(N_{c}=L_{c}/T, where L_{c} is the length of the genome sequence c, T (theoretical ultimate precision) is the fragment size which can be detected theoretically; and when the window size is W, the sliding length of the windows is S, and the number of each group in the run test is N, the theoretical ultimate precision T=W+S*N); and in the set of the candidate breakpoints, the minimum p value is the termination p value (p_{final}) of the genome sequence.

In some particular embodiments of the method of the present invention, the step of performing a confidence-based selection on fragments between said genetic variation sites is: for a fragment between genetic variation sites on the reference genome sequence, the average of Z_{i,j} in the fragment is calculated and recorded as Z̅, wherein if Z̅ of the fragment is smaller than −1.28, then the fragment is a fragment deletion, and if same is greater than 1.28, then the fragment is a fragment duplication.

In the present invention, the run test is a non-parametric test in which, according to the uniform state of distribution of elements in two groups after mixing of the two groups, acquires the significance P value to evaluate these two groups. See http://support.sas.com/kb/33/092.html.

In the present invention, during the test with the control sample as the test sample, sequencing or experiments in practice will lead to the existence of differences in the number of fragments for sequencing to which different fragments aligned in the whole genome, so in the test process, these differences will be discriminated, and just fragments at both ends of a breakpoint have not yet reached the variation level. At the beginning of the test, these differences cannot be discriminated relatively significantly by the candidate breakpoints, so an N value is needed to be defined, which ensures that when the number of the breakpoints is the N value, the experiments can discriminate these differences relatively well, and then it can be more precise to use the threshold obtained here in testing the test sample.

In the present invention, for the determination of the threshold of the Z̅ value: statistics is performed on the control sample according to steps a) and b), and then the Z value in each window meets the normal distribution, and −1.28 and 1.28 are quantiles where the cumulative probability in the normal distribution is 0.05 and 0.95, respectively. According to the need, those skilled in the art can also select the Z̅ value as a value with a greater absolute or a smaller absolute, which correspond to a greater cumulative probability and a smaller one in the normal distribution, respectively; however, −1.28 and 1.28 are the most preferred thresholds established for the present invention by the inventors through a large number of experiments, and a threshold with a greater absolute other than the two values will increase the false negative/false positive rate in a detection result.

In an application of the method of the present invention, e.g. non-invasive fetal CNV screening on a suitable population is conducive to providing genetic counseling and providing a basis for clinical decision making; and prenatal diagnosis can effectively prevent the birth of an infant patient. The suitable population of the present invention can be all healthy pregnant women, and examples of the suitable population are only used to describe the present invention, and should not limit the scope of the present invention.

The following will detail the embodiments of the present invention in conjunction with examples, but it will be understood by those skilled in the art that the following examples are only used to describe the present invention, and should not be considered to limit the scope of the present invention.

Those without indicated specific conditions in the examples are performed according to the routine conditions or the conditions proposed by the manufacturers. Reagents or instruments used without indicated manufacturers are all routine products available through the market. The manufacturer's article number of each reagent or kit is in the following brackets. The adapters and tag sequences used for sequencing are derived from the Multiplexing Sample Preparation Oligonutide Kit of the Illumina Corporation.

### Example I

### Detection of Fetal Large-Fragment Copy Number Variation in 1 Case of Maternal Plasma, and Detection of Fetal Aneuploidy Variation in 9 Cases of Maternal Plasma

1. DNA extraction:

According to the operational flow for TiangenDP327-02Kit, DNA was extracted from the above-mentioned 8 cases of plasma samples (see Table 1 for sample Nos.), a library was constructed for the extracted DNA according to the modified Illumina/Solexa standard library construction flow, adapters used for sequencing were added to both ends of DNA molecules with main bands concentrated at 200 bp, a different tag sequence was added to each sample, and then hybridization with complementary adapters on the flowcell surface was performed. A layer of single-chain primers were linked through the flowcell surface, and after turning into single chains, DNA fragments were “fixed” at one end on the chip through complementation with primer bases on the chip surface; and the other end (5′ or 3′) was randomly complementary to another nearby primer and was also “fixed” to form a “bridge”, amplification was repeated for 30 runs, and each single molecule was amplified by about 1,000 times to form a monoclonal DNA cluster. Then through pair-end sequencing on IlluminaHiseq2000, DNA fragment sequences of about 50 bp in length were obtained.

Specifically, about 10 ng of DNA obtained from the above-mentioned plasma samples was subjected to the modified Illumina/Solexa standard library construction flow, and see the product instructions for the specific flow (the Illumina/Solexa standard library construction instructions provided by http://www.illumina.com/). The size of the DNA library was determined and the inserted fragments were determined to be about 200 bp via 2100Bioanalyzer (Agilent), and on-computer sequencing could be performed after precise quantification by QPCR.

2. Sequencing: in this example, the DNA samples obtained from the above-mentioned 10 cases of plasma were manipulated according to the instructions for ClusterStation and Hiseq2000 (PEsequencing) officially published by Illumina/Solexa to obtain the data amount of about 0.36 G from each sample to perform on-computer sequencing, and each sample was discriminated according to said tag sequences. The DNA sequences obtained by sequencing were aligned with the human genomic reference sequence build 36 in the NCBI database (hg18; NCBIBuild 36) in the manner of no mismatch allowed using the alignment software SOAP2 (obtained from soap.genomics.org.cn) to obtain locations of the DNA sequences for sequencing on said genome.

3. Data analysis

a) Calculating the relative number of reads for the test sample: the length of the reference unique reads was selected as 50 bp, the number of the reference unique reads was counted, the human genomic reference sequence was divided into windows with the same number of reference unique reads (840,000), the average of all the window sizes was 1 Mb, and the distance between adjacent windows was S=10 kb. The actual number of reads, r_{i,j}, falling in each window in the above-mentioned step 2 was counted, where the subscripts i and j represented the serial number of the window and the serial number of the sample respectively, and the GC content of each window, GC_{i,j}, was calculated, and the relative number of reads,

$${R}_{i , j} = {log}_{2} ( \frac{{r}_{i , j}}{{\overline{r}}_{j}} ) ,$$

was calculated, where the average number of reads is

$${\overline{r}}_{j} = \frac{1}{n} {\sum }_{i = 1}^{n} {r}_{i , j} ;$$

b) Data correction and standardization:

\textcircled{1} in a coordinate system with the GC content as the abscissa and the relative number of reads R as the ordinate, performing linear fitting on R_{i,j} and GC_{i,j} of the control sample to obtain the slope aᵢ and the intercept bᵢ,

\textcircled{2} for each window of the test sample, calculating the corrected relative number of reads \tilde{R}_{i,j}=aᵢ×GC_{i,j}+bᵢ,

\textcircled{3} for each window of the test sample, calculating the standardized relative number of reads Z_{i,j}:

Z_{i,j}=(R_{i,j}−\tilde{R}_{i,j}−meanᵢ)/SDⱼ,

where

$${mean}_{j} = \frac{1}{n} {\sum }_{i = 1}^{n} ( {R}_{i , j} - {\tilde{R}}_{i , j} ) , \\ {SD}_{j} = \sqrt{\frac{1}{n - 1} {\sum }_{i = 1}^{n} {( {R}_{i , j} - {\tilde{R}}_{i , j} - {mean}_{j} )}^{2}}$$

c) Window merging

\textcircled{1} initialization: the position of the starting point of each window on the reference genome sequence was recorded as the position of the statistic Z. Then corresponding to the chromosomal position on the reference genome, the Z value had a change trend. The position corresponding to the inflection point of the Z value (i.e. a critical point where the Z value is converted from an increasing trend into a decreasing trend or turns from a decreasing trend into an increasing trend) was found. For any chromosome, positions with the distance therebetween of at least 100 windows were then selected sequentially starting from the starting point of the first window, and these positions were recorded as candidate breakpoints bₖ (k=1, 2, . . . , s; s is an integer>0);

\textcircled{2} optimal iteration: in order to study copy number variation analysis or aneuploidy in any chromosome in the genome (in this example, only human chromosomes 1-22 were studied), all sorted candidate breakpoints of each chromosome were recorded as B_{c}={b₁, b₂, . . . , bₛ}, wherein two fragments, one on the left and the other on the right, exist for each candidate breakpoint bₖ and said fragments were a region from the previous breakpoint to the breakpoint and a region from the breakpoint to the next breakpoint; the p value (pₖ) obtained by subjecting all Z_{i,j} in these two fragments to the run test was regarded as “the significance of bₖ as a breakpoint”; a candidate breakpoint with the maximum pₖ was excluded; and this step was repeated until all p values were smaller than the termination p value (p_{final}) for the chromosome;

\textcircled{3} acquisition of the termination p value: in the test process, the above-mentioned steps a) to c) \textcircled{1} were performed by using the control sample as the test sample, for the chromosome c, all sorted candidate breakpoints of the cth chromosome were recorded as B_{c}={b₁, b₂, . . . , bₛ}, wherein two windows, one on the left and the other on the right, exist for each candidate breakpoint bₖ; the p value (pₖ) obtained by subjecting all Z_{i,j} in these two windows to the run test was regarded as “the significance of bₖ as a breakpoint”; a candidate breakpoint of the least significance was excluded, until the number of candidate breakpoints was equal to the expected value N_{c} (N_{c}=L_{c}/T, where L_{c} is the length of the chromosome, and the theoretical ultimate precision T=2 Mb); and in the set of the candidate breakpoints, the minimum p value was the termination p value (p_{final}) of the chromosome, see the following table;

Relevant Values Used in the Example

| | | | |
|---|---|---|---|
| 20 | 62,435,964 | 31 | 1.56E−95 |
| 21 | 46,944,323 | 23 | 3.96E−89 |
| 22 | 49,691,432 | 24 | 2.38E−111 |

d) fragment filtration after window merging: in order to further filter fragments obtained after window merging, the average of Z_{i,j} in the fragment was calculated and recoded as Z̅, and if Z̅ of the fragment was smaller than −1.28 or greater than 1.28, then the fragment was a copy number variation. See Table 1 for the results.

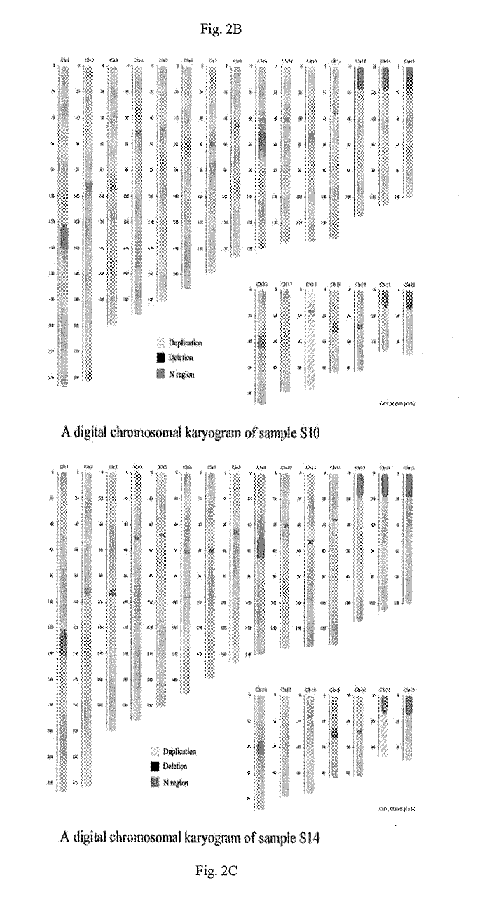

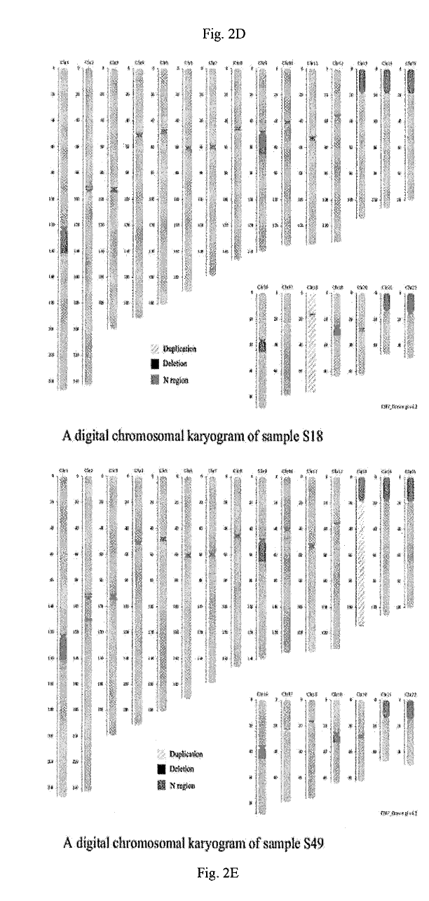

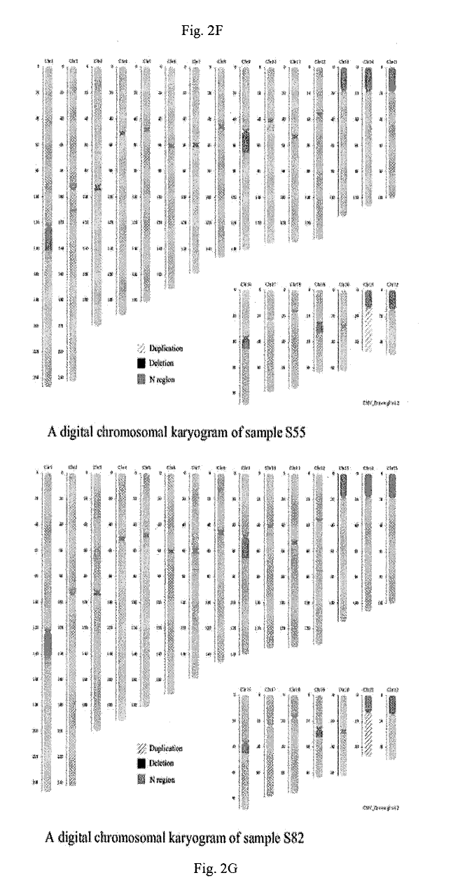

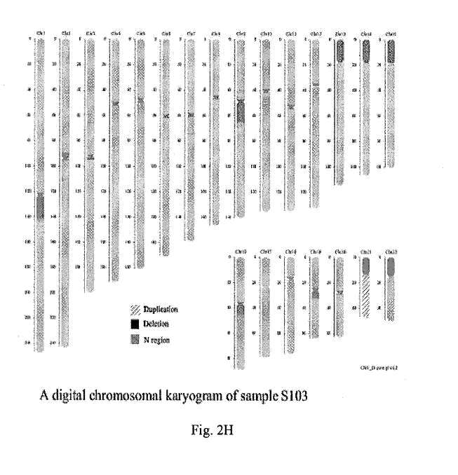

4) Result visualization, see FIG. 2.

TABLE 1
A list of CNV results of all samples in the implementation case

| | | CNV starting | CNV ending | CNV | Judgment | Regions and |
|---|---|---|---|---|---|---|
| No. | Chromosome | point | point | size | result | bands involved |
| S67 | 4 | 181,243,323 | 191,250,465 | 10.1M | Deletion | 4q34.3→q35.2 |
| | 7 | 34,983 | 17,074,358 | 17M | Duplication | 7p21.1→p22.3 |
| S10 | 18 | 1 | 76,117,153 | 76.1M | Duplication | 18p11.32→q23 |
| S14 | 21 | 1 | 46,944,323 | 46.9M | Duplication | 21p13→q22.3 |
| S18 | 18 | 1 | 76,117,153 | 76.1M | Duplication | 18p11.32→q23 |
| S49 | 13 | 1 | 114,142,980 | 114.1M | Duplication | 13p13→q34 |
| S55 | 21 | 1 | 46,944,323 | 46.9M | Duplication | 21p13→q22.3 |
| S82 | 21 | 1 | 46,944,323 | 46.9M | Duplication | 21p13→q22.3 |
| S103 | 13 | 1 | 114,142,980 | 114.1M | Duplication | 13p13→q34 |

The results of CNV analysis in the present invention were compared with the results by the CGH chip in the following, and the comparison results are shown as the following Table 2. For the results by the CGH chip, the Human Genome CGH Microarray Kit (Agilent Technologies Inc.) was used.

Same was obtained according to the protocol by the provider, and the steps are briefly described as follows:

DNA from a healthy person with the same sex as the sample to be tested or mixed DNA from male and female healthy persons was used as the reference DNA, the reference DNA and the DNA to be tested were labeled with the fluoresceins, Cy3 and Cy5, respectively, and then hybridized with probes, and if the fluorescence intensity ratio of the DNA to be tested to the reference DNA was 1, then it could be understood as that the amounts of the DNA to be tested and that of the reference DNA were equal, and if the ratio was not equal to 1, then it was indicated that there is deletions or amplifications in the DNA to be tested. The resolutions of various types of Array CGH depend on the interval between and the length of the probes on the microarray. Flow: remaining cell culture liquid after the G-banding chromosome examination was collected, and genomic DNAs of the sample to be tested and that of the control sample were extracted. After purification, the sample to be tested and the reference sample were fluorescently labeled differently, and then the samples were mixed with Cot-1 DNA blocking non-specific hybridization, denatured, pre-annealed, and hybridized with the microarray, and finally, DNA that did not bind to the microarray in a target specific manner was eluted, and the fluorescence intensity ratio of the two signals on the target on each microarray was obtained after scanning and analysis by software, which reflected the change in the copy number of a corresponding sequence or gene between the genomic DNA of the sample to be tested and the genomic DNA of the reference sample.

TABLE 2
Comparison of the detection results of the example of
the present invention and the results by the CGH chip

| | | | Detection results of | |
|---|---|---|---|---|
| | | Results by the | the method of the | Judgment |
| No. | Chromosome | CGH chip | present invention | result |
| S67 | 4 | Deletion: | Deletion: | Consistent |
| | | 181,170,528- | 181,243,323- | |
| | | 190,958,960 | 191,250,465 | |
| | 7 | Duplication: | Duplication: | Consistent |
| | | 204,709- | 34,983- | |
| | | 16,283,844 | 17,074,358 | |

The results of CNV analysis in the present invention were compared with the results of standard karyotyping in the following, and the comparison results were shown as the following Table 3. The steps of standard karyotyping were as follows:

(1) The amniotic fluid obtained by centesis was centrifuged for 5 minutes (at a rotational speed of 800-1,000 revolution/minute), and then inoculated in an inoculation hood. The supernatant was pipetted out and retained for other examinations, 0.5 ml of amniotic fluid and precipitated amniotic fluid cells remained in the centrifuge tube, and the precipitated fetal exfoliated cells and amniotic cells were pipetted uniformly into a cell suspension, and inoculated into three culture flasks containing a culture solution.

(2) The culture flasks were placed in a carbon dioxide incubator.

(3) 5-7 days after inoculation, viable cells in the amniotic fluid were adhered to the bottoms of the flasks, and began to grow, and the growth status of the cells could be observed with an inverted microscope. If adherence had occurred, the culture solution could be changed, 3-5 ml of the fresh culture solution was added, and the solution was changed once every 2-3 days afterwards. Adherent cells included epithelioid cells, fibroblast-like cells and amniotic fluid cells which were a kind of cells with the morphology falling between the epithelioid cells and fibroblasts. The above-mentioned three kinds of cells all formed clones, and if the growth status was good, 11-14 days after inoculation, there could be more than ten large-flake clones at the bottoms of the flasks, unaided eyes could also see flaky clones at the bottoms of the flasks, and the cell nuclei were large and round. At this time, smear preparation, or referred to as harvest, could be prepared. One day before harvest, same should be changed with the fresh culture solution to increase nucleus division.

(4) Harvest: harvest was performed 14-20 days after culture on average. Colchicine of 0.04 nanogram/milliliter was added into the culture flasks, the cells were made to be halted at the metaphase and cultured for 5-15 hours, and it could be seen under an inverted microscope that there were many cell nuclei at the mitotic phase, and the cells were round and large and bright as a flake of bright pearls, and were interlinked. The amount of colchicine added could be different in various laboratories.

(5) Trypsinizing: the culture solution in the culture flasks was poured into centrifuge tubes, 0.5 ml of 0.02% EDTA trypsin digestion solution or 0.5 ml of 0.15% pronase was placed at the bottoms of the culture flasks, the cell clones at the bottoms of the flasks were pipetted gently with a long curved glass pipette, it was seen under an inverted microscope that clone cells had floated, same was pipetted into the centrifuge tubes, and then, cells that had not yet floated were washed with 0.5-1 ml of Hank's solution, continued to be pipetted with the long pipette and were poured into the centrifuge tubes after being made to be completely detached. Centrifugation was performed for 5 minutes at a speed of 800-1,000 revolution/minute, the supernatant was removed and the cells were reserved for use.

(6) Hypotonic treatment: 4 ml of 0.075M KCl solution at 37° C. was added gently into the above-mentioned centrifuge tubes and cells, the bottoms of the tubes were flicked gently with a finger or the precipitated cells were dispersed with a sharp pipette, same were placed in a 37° C. water bath for 16 minutes (the time of hypotonic treatment could be determined according to their own experiences in various laboratories), and centrifuged for 5 minutes, the supernatant was removed, the fresh fixation solution (methanol:glacial acetic acid=3:1) was dropped gently in along the walls of the tubes, the bottoms of the tubes were patted gently with a finger, the cells were separated uniformly, fixed for 15 minutes and centrifuged, the fixation solution was changed, and after second-time fixation was performed for 30 minutes, same overnighted.

(7) Smear blowing: after centrifugation, the supernatant was removed and 0.5 ml was retained and prepared into a cell suspension, or the supernatant was completely removed and 0.5 ml of newly-prepared fixation solution was added, and after same is pipetted carefully with a long and thin glass tube, one drop was pipetted out, dropped onto a glass slide taken out from ice water, and dispersed by blowing gently, and after the glass slide was placed in the air and dried, the dispersion status of chromosomes was observed under a microscope, and then smear blowing was continued. The dried glass slide could be directly stained with Giemsa.

(8) Banding: if the chromosomal morphology was good, Giemsa banding, referred to as G-banding, could be performed. The glass slide was firstly baked at 65° C. for 1 hour, or baked at 37° C. for 24 hours, the glass slide was placed in 0.25% trypsin solution for 20-25 seconds at room temperature, subjected to physiological saline twice, placed in 2% Giemsa solution for 5-10 minutes, taken out, washed with running water, and dried in the air, and the chromosomes could be observed under a microscope to perform karyotyping.

TABLE 3
Comparison of the detection results in the present implementation
case and the standard karyotyping detection results

| | | Detection results of the | |
|---|---|---|---|
| | Standard | method of the present | Judgment |
| No. | karyotyping | invention | result |
| S10 | T18 | T18 | Consistent |
| S14 | T21 | T21 | Consistent |
| S18 | T18 | T18 | Consistent |
| S49 | T13 | T13 | Consistent |
| S55 | T21 | T21 | Consistent |
| S82 | T21 | T21 | Consistent |
| S103 | T13 | T13 | Consistent |

Although the particular embodiments of the present invention have been detailed, it will be understood by those skilled in the art that according to all teachings that have been disclosed that those details can be subjected to various modifications and substitutions, and these changes are all within the scope of protection of the present invention. The full scope of the present invention is given by the appended claims and any equivalent thereof.

1. A method for detecting genetic variation, comprising the following steps:
1) acquiring reads from a test sample;
2) aligning said reads with a reference genome sequence;
3) dividing said reference genome sequence into windows, calculating the number of reads which are aligned to each window, and acquiring the statistic for each window on the basis of the number of said reads; and
4) for a fragment of the reference genome sequence, on the basis of the change in the statistics of all the windows thereon in the fragment of the reference genome sequence, acquiring positions where a significant change occurs in statistics of the windows on both sides, these positions being positions where genetic variation sites of the test sample are on the reference genome sequence.

2. The method of claim 1, further comprising the following step:
5) screening the genetic variation sites to obtain post-screening genetic variation sites.

3. The method of claim 1, wherein the length of said reads is 25-100 nt.

4. The method of claim 1, wherein the number of said reads is at least 1 million.

5. The method of claim 1, wherein said windows have the same number of the reference unique reads.

6. The method of claim 1, wherein said windows have an overlap or have no overlap therebetween.

7. The method of claim 1, wherein said statistic approximately fits normal distribution obtained by the standardization processing on the number of reads which are aligned to a window.

8. The method of claim 7, wherein said standardization is based on the average number of reads which are aligned to all the windows.

9. The method of claim 1, wherein said genetic variation site is the median point between an inflection point where said statistic turns from ascending to descending and the next same inflection point, and there is at least 50, at least 70, at least 100, preferably 100 window lengths between two genetic variation sites.

10. The method of claim 2, wherein said step 5) is:
for each genetic variation site, performing statistics on the difference between two numerical groups consisting of statistics of windows contained in the fragment between the genetic variation site and its preceding genetic variation site and in the fragment between the genetic variation site and its subsequent genetic variation site, and removing the genetic variation site whose significance value of difference is maximum and greater than a preset threshold; and repeating the above-mentioned process, until the significance values of difference of the genetic variation sites are all smaller than the preset threshold.

11. The method of claim 10, wherein said significance of difference is performed by the run test, removing the genetic variation site whose significance value in the run test is maximum and greater than the preset threshold; and repeating the above-mentioned process, until the significance values of the genetic variation sites in the run test are all smaller than the preset threshold.

12. The method of claim 10, wherein said preset threshold is acquired by the following steps:
a) acquiring the genetic variation sites according to the method of claim 1 by substituting the test sample with a control sample,
b) for each genetic variation site, performing statistics on the difference between two numerical groups consisting of statistics of windows contained in the fragment between the genetic variation site and its preceding genetic variation site and in the fragment between the genetic variation site and its subsequent variation site, and removing the genetic variation site which is the least significant; and
c) repeating the above-mentioned step b), until the number of remaining candidate breakpoints is equal to the expected value N_{c}, wherein N_{c}=L_{c}/T, L_{c} is the length of the genome sequence, the theoretical ultimate precision T is the fragment size which can be detected theoretically, the theoretical ultimate precision T=W+S*N when the average of the window sizes is W, the sliding length of the windows is S and the number of each window group in the run test is N, and among significance values of all the remaining candidate breakpoints, the minimum is the significance threshold.

13. The method of claim 1, further comprising:
performing a confidence-based selection on fragments between said genetic variation sites.

14. The method of claim 13, wherein performing a confidence-based selection on fragments between the genetic variation sites comprises:
i) calculating the distribution probability of the statistics through the distribution pattern of the statistics for the windows, and setting a threshold; and
ii) comparing the average of the statistics of windows in the fragment between post-screening genetic variation sites with said threshold, and determining whether the fragment between the genetic sites is anomalous on the basis of the comparison result.

15. The method of claim 14, wherein performing a confidence-based selection on fragments between the genetic variation sites comprises:
i) calculating the distribution probability of the statistics through the distribution pattern of the statistics for the windows, and setting a first threshold and a second threshold; and
ii) comparing the average of the statistics of windows in the fragment between post-screening genetic variation sites with said first threshold and second threshold,
wherein if the statistics for windows in the fragment are smaller than the first threshold, the fragment is a fragment deletion, and if same are greater than the second threshold, the fragment is a fragment duplication.

16. The method of claim 15, wherein said first threshold is a value of the statistic where the cumulative probability is 0.05, and/or said second threshold is a value of the statistic where the cumulative probability is 0.95.

17. A computer-readable medium, carrying a series of executable codes, which can execute the method of claim 1.

18. A method for detecting fetal genetic variation, comprising:
acquiring a maternal sample containing fetal nucleic acid;
sequencing said maternal sample; and
detecting the genetic variation using the method of claim 1.

19. The method of claim 18, wherein said maternal sample is maternal peripheral blood.

20. The method of claim 3, wherein the length of said reads is 35-100 nt.
